(19) United States
(12) Patent Application Publication
(10) Pub. No.: US 2026/0179604 A1
(43) Pub. Date: Jun. 25, 2026

(54) SYSTEM AND METHOD OF SEGMENTING AUDIO DATA FOR TRANSCRIPTION

(71) Applicant: Doximity, Inc., San Francisco, CA (US)

(72) Inventors: Emmanuel Lagarrigue Lazarte, Bahía Blanca (AR); Juan Alejandro Giannuzzo, Alta Gracia (AR); Aaron Arthur Gray, Trussville, AL (US); Elizabeth Doris Marie Stephenson, Port Saint Lucie, FL (US); Ronnie Christian Rocha, San Antonio, TX (US)

(21) Appl. No.: 18/990,643

(22) Filed: Dec. 20, 2024

(51) Int. Cl. G10L 15/04; G10L 15/26; G16H 10/60

(52) U.S. Cl. CPC G10L 15/04; G10L 15/26; G16H 10/60

(57) ABSTRACT

A system performs a method including generating audio data corresponding to a medical visit. In addition, the system may receive a duration value. The system may segment the audio data into several audio data segments having a duration equal to or greater than the duration value.

### TECHNICAL FIELD

This disclosure generally relates to audio recordings. In particular, this disclosure relates to transferring audio data of audio recordings for transcription.

### BACKGROUND

Physicians take notes during patient visits to track patient progress, ensure continuity of care, and facilitate communication with other healthcare providers. Traditionally, physicians have written notes manually, but notes may now be transcribed from voice recordings of patient visits. Transcription of voice recordings can both save time and enhance accuracy and detail of the visit summary. The voice recordings can be transmitted by the physician to a remote transcription service that returns notes for the physician to update and save in an electronic health record (EHRs) of a patient, for example.

### BRIEF DESCRIPTION OF THE DRAWINGS

Embodiments and implementations of the present disclosure will be understood more fully from the detailed description given below and from the accompanying drawings of various aspects and implementations of the disclosure, which, however, should not be taken to limit the disclosure to the specific embodiments or implementations, but are for explanation and understanding only.

FIG. 1 is a diagram illustrating an example network architecture in accordance with one or more embodiments of the disclosure.

FIGS. 2A-2C are pictorial views of a user interface for recording a medical visit in accordance with one or more embodiments of the disclosure.

FIG. 3 is a flow diagram of operations performed by a system during a method of transferring audio data in accordance with one or more embodiments of the disclosure.

FIG. 4 is a flow diagram of interactions in a system during audio data transfer in accordance with one or more embodiments of the disclosure.

FIG. 5 is a flow diagram of operations performed by a mobile device during a method of transferring audio data in accordance with one or more embodiments of the disclosure.

FIG. 6 is a pictorial view of a user interface for generating notes based on audio data recording a medical visit in accordance with an embodiment of the disclosure.

FIG. 7 is a pictorial view of a user interface for displaying notes generated based on audio data recording a medical visit in accordance with an embodiment of the disclosure.

FIG. 8 is a block diagram of an example computing device that may perform one or more of the operations described herein, in accordance with some embodiments.

### DETAILED DESCRIPTION

The process of having audio recordings transcribed typically begins with a physician recording a patient visit using a mobile device. The recording is sent by the physician as an audio file to a remote transcription service, e.g., via a software application that runs during the patient visit. The transcription service then processes the audio file, converting the spoken words into written text manually or using speech recognition technology. The transcribed text can be returned to the physician, e.g., via the software application or email. The physician can then review, update, and save the text notes in a storage system, such as a medical chart or an electronic health record (EHR) system.

Existing methods of having audio recordings transcribed to support physician notetaking have several drawbacks. First, the transfer of audio files may be performed during or after a patient visit and involves a file transfer to the remote transcription service. The file transfer is subject to error, however. For example, when transferring over a poor connection or when a remote server of the remote transcription service is not operational, disruptions in transfer can occur. If the disruption occurs during the course of the patient visit, the entire transcription may be corrupted and lost. Additionally, transfer of the audio file and/or transcription may occur in a bulk manner, which can result in a time-consuming delay in receiving the transcription. For example, the transfer of the audio file, transcription of the recording, and return of the transcription can take several minutes, e.g., 5-15 minutes, under typical circumstances for a half-hour to an hour patient visit. The process can therefore be time-consuming and may be perceived by the physician as inefficient and laggy. Accordingly, there is a need for methods of generating and transferring audio data in a manner that facilitates stable and efficient transcriptions.

In an aspect, audio data is cached, e.g., temporarily stored as audio files, to support stable transcriptions. More particularly, the audio data can be partitioned into chunks, which can be approximately one minute long, and the chunks are transmitted sequentially to a server for use in transcribing, creating, and saving notes. The chunks are cached locally during the transmission process, while audio is being recorded. In the event of a disrupted connection between the mobile client and the server, the cached chunks can be stored. Storage of the chunks is performed locally while a reconnection is attempted. When a reconnection is established, the stored chunks are then transmitted to the server for note generation. Accordingly, caching data ensures that when a disconnection occurs, the source data used for note generation is not lost.

In an aspect, the chunks of audio data that are transmitted to the server are partitioned in a particular manner that promotes clarity of the transcribed data and supports speed of transcriptions. For example, the chunks are divided at times that ensure that important words or phrases, such as “cancer,” are not severed or lost in transmission, e.g., being transmitted and transcribed as the two words “can” and “sir.” Partitioning can be triggered by various techniques including silence detection. Upper limits can be placed on a timeframe to perform the segmentation, ensuring that chunking will continue when the call is placed in a noisy environment, such as with background music. Furthermore, transferring chunks can allow for transcriptions to be returned quickly, e.g., within 20 seconds of the patient visit completing. Accordingly, audio data chunking provides accurate transcriptions that are perceived by the physician as being quickly and efficiently produced.

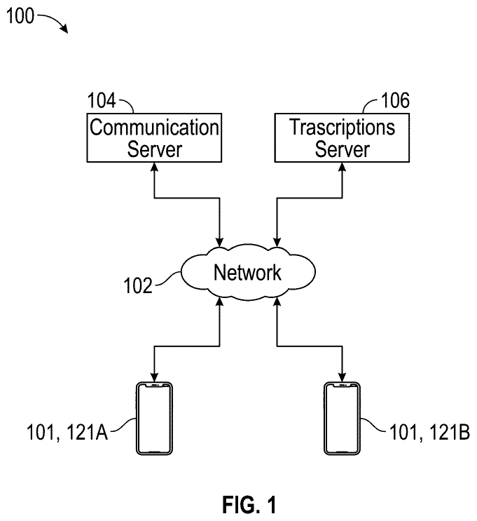

FIG. 1 is a diagram illustrating an example network architecture, in accordance with one or more embodiments of the disclosure. A network architecture 100 includes a network 102 interconnecting one or more computing devices 101, e.g., mobile device(s), with one or more other system components, including a communication server 104 and/or a transcription server 106. More particularly, the system components can be in data communication with each other through the network 102.

Examples of computing devices 101 may include, but are not limited to, a mobile device, e.g., a smartphone, a tablet computer, or a laptop computer, or a desktop computer, etc. A first mobile device 121A can be a smartphone of a first healthcare provider, e.g., a physician, and a second mobile device 121B can be a smartphone of a second healthcare provider, e.g., a physician assistant. The various computing devices 101 can operate and communicate simultaneously, e.g., to record a patient visit simultaneously, and can have different credentials that communicate with the servers at the simultaneously.

Healthcare providers may be people who provide health related services and/or products to the user. Examples of healthcare providers may include, but are not limited to, doctors, pharmacists, dentists, nurses, therapists, psychologists, technicians, surgeons, etc. Each healthcare provider may use a computing device (e.g., smartphone, tablet computer, etc.) to manage visits with patients. For example, the healthcare provider(s) can use the mobile devices to record conversations with patients during patient visits.

As described below, the recordings generated by the mobile device 101 of the healthcare provider can be transmitted to the communication server 104. The communication server 104 can communicate the recordings to the transcription server 106 to have the recordings transcribed. Transcriptions may be returned to the communication server 104 and, optionally, communicated to the mobile device 101 for display to the healthcare provider. Furthermore, the communication server 104 may query the transcription server 106, e.g., with system or user prompts, to cause artificial intelligence programs running on the transcription server to return specific information related to the recordings. Accordingly, the network architecture supports processes to allow the healthcare provider to record patient visits and to receive information, e.g., transcriptions and/or notes summarizing the transcriptions, related to the patient visits.

Processes described below may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the processes may be performed by the various components of the system, including the servers and mobile devices described above.

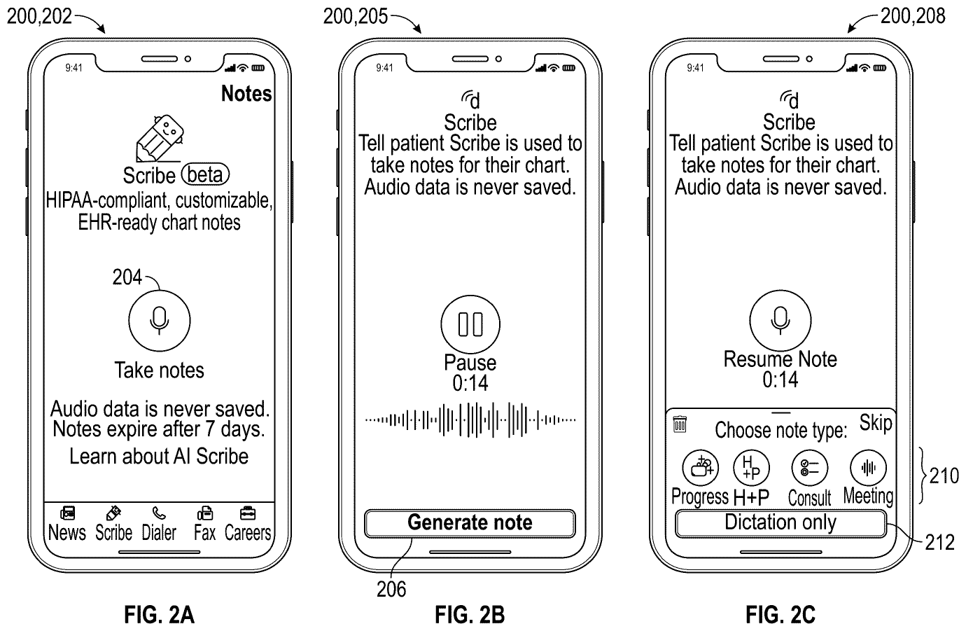

FIGS. 2A-2C are pictorial views of a user interface 200 for recording a medical visit in accordance with one or more embodiments of the disclosure. The user interface 200 can allow a healthcare provider to interact with a mobile application running on the mobile device 101. The mobile application can, for example, be a medical social network application that helps the healthcare provider perform day-to-day professional functions, such as taking notes to document a medical visit.

Referring to FIG. 2A, a user interface 200 can include a tab to enable a recording and/or notes to be generated to memorialize a medical visit, such as a meeting between the healthcare provider and a patient. The user interface 200 can include a first screen 202 indicating to a user that the functionality is HIPAA-compliant and will not result in the storage of audio data for more than a period of time. The first screen 202 can include a record element 204, e.g., a microphone icon, which can initiate a recording of the medical visit when pressed by the user.

Referring to FIG. 2B, in response to receiving a user selection of the record element 204, the user interface 200 can transition to a second screen 205. The second screen 205 can indicate that the medical visit is being recorded and provide instructions to the user in relation to the visit. For example, the second screen 205 can instruct the physician to inform the patient that the mobile application is used to generate audio data and take related notes for a patient chart, and that the audio data is not saved. The second screen can include additional control elements, such as a pause button, to allow the recording to be paused and resumed by the user throughout the visit. The second screen may also indicate a duration of the ongoing recording, e.g., 14 seconds.

In an embodiment, the second screen includes a control element to enable a note to be generated based on the recorded medical visit. For example, the user interface 200 can include a note element 206 to receive a user input for initiating the generation of the note. More particularly, the user can press the note element 206 to end recording and present the user with options for note types.

Referring to FIG. 2C, from the second screen 205, the user interface 200 can transition to a third screen 208 in response to the user selection of the note element 206. The third screen 208 can present one or more selectable note icons 210 indicating different types of notes that can be generated based on the audio recording of the medical visit. Selection of one of the icons can cause the mobile application to generate the indicated type of note. For example, the mobile application can generate a note (or receive the note generated by the communication server 104) to display to the user. Optionally, the user interface 200 can include a dictation element 212 to allow the user to generate dictation only, e.g., a transcript of the conversation with the patient. More particularly, the system can generate the transcript of the conversation in response to a user selection of the dictation element 212. The transcript may be modified, e.g., the transcript of the conversation may be altered for readability and may not exactly replicate the conversation.

The user interface screens described above allow the user to interact with the mobile application during the medical visit to generate audio data and a corresponding transcription. The transcription can include a direct transcription or a summary of such transcription, e.g., notes summarizing the conversation. Computer-implemented methods related to the user interactions are described in more detail below. The combination of user interactions and computer operations allow the user to generate and save, for access by the mobile device 101 or another computing system, note content that summarizes the medical visit.

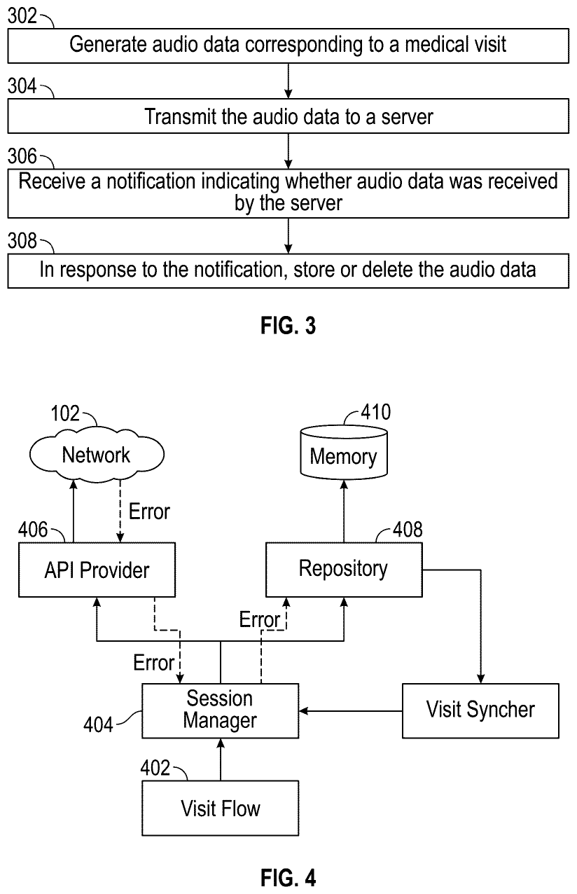

FIG. 3 is a flow diagram of operations performed by a system during a method of transferring audio data in accordance with one or more embodiments of the disclosure. At operation 302, audio data is generated corresponding to the medical visit. Such generation is illustrated with respect to FIGS. 2A-2B, in which selection of the record element 204 activates recording hardware and software on the mobile device 101, causing a microphone of the mobile device 101 to record a conversation between a physician and a patient. More particularly, selection of the record element 204 can allow microphone access on the mobile device 101.

FIG. 4 is a flow diagram of interactions in a system during audio data transfer in accordance with one or more embodiments of the disclosure. A visit flow block 402 refers to the user interactions and mobile device activation that occurs through activation of the record element 204. The visit flow block 402 communicates with a session manager block 404. The session manager can manage mobile device operations, such as activating one or more applications or components and creating an environment within which the generation of audio data can take place. For example, the session manager can turn on the microphone, generate audio data, and post the audio data to other system components. The session manager may divide the recorded audio into chunks, as described in more detail below.

At operation 304, the recorded audio data is transmitted to a server, e.g., the communication server 104. The session manager can send one or more chunks of audio data, e.g., one or more audio files containing recorded audio data representing the medical visit, to an API provider block 406. The one or more chunks of audio data may be used by the transcription server 106 to create a transcript of the medical visit. The API provider block 406 can be a component of the system that communicates with the communication server 104 through the network 102. Accordingly, the one or more chunks of audio data can be relayed by the API provider from the session manager to the communication server 104 via the network 102.

Notably, at the time that the session manager provides the one or more chunks of audio data to the API provider, the session manager may also create a copy of the one or more chunks of audio data. The copies may be stored as described below. More particularly, the one or more chunks of audio data can be stored locally on the mobile device 101 when the data is transmitted to the remote communication server 104. The one or more chunks of audio data can be stored, for example, in a repository 408 on the mobile device 101. The repository 408 may, for example, be a local cache of the mobile device 101. The one or more chunks of audio data can be cached (stored temporarily) as opposed to being stored permanently or indefinitely. More particularly, cached audio data may be stored until audio data is successfully transferred to the API provider, at which time the audio data may be deleted from the cache. Similarly, even in cases when the audio data does not successfully transfer to the API provider, the audio data can be deleted from the cache after a time period.

Existing audio data transfer methods do not store transmitted audio data locally and, thus, if the transmission fails then an error message may be presented to the user and the audio data may be lost. By contrast, the method of breaking the flow into two parts—a transmission of the audio data and a local caching of the audio data—can avoid the risk of losing data. Whether and for how long the locally cached audio data is stored by the mobile device 101 can, however, depend on communications from the communication server 104.

At operation 306, a notification is received indicating whether the audio data was received by the server. The communication server 104 can send the notification, in response to the attempted transmission of the one or more chunks of audio data. The transmission between the API provider mobile device 101 and the communication server 104 can include posting audio data chunks through the network 102. When the audio data chunks are posted, the notification can indicate that the upload of the chunk succeeded. When the audio data chunks are not posted, for example, because the connection between the mobile device 101 and the communication server 104 fails, the notification can indicate that the upload of the chunk failed.

At operation 308, in response to the notification, the audio data is either stored or deleted by the mobile device 101. In an embodiment, the audio data is deleted in response to the notification indicating that the audio data was received by the server. For example, the notification can indicate that the chunk is uploaded successfully, and the audio data copy at the mobile device 101 is no longer needed. In such case, the audio data chunk cached in the repository 408 can be deleted and removed from the local storage 410 of the mobile device 101.

In an alternative embodiment, the audio data is stored in response to the notification indicating that the audio data was not received by the server. For example, the notification can indicate that the chunk is not uploaded successfully, and the audio data copy is needed to allow a transcription to be created. The audio data can be stored until the transmission can be repeated, e.g., when a connection between the mobile device 101 and the communication server 104 is reestablished, and completed.

Audio chunks can be stored locally, e.g., in the repository 408, or in another memory 410 of the mobile device 101. The memory 410 can be a long-term storage (as opposed to temporary storage) of the mobile device 101. Transmission can be repeatedly attempted until the audio chunks are successfully posted. Accordingly, the overall process flow can involve storing the audio chunks in progressively longer term memory 410 of the mobile device 101 until the transmission succeeds.

The audio data can be transmitted to the server again in response to the notification indicating that audio data was not received by the server. More particularly, in response to receiving the notification indicating the transmission error, the session manager can retrieve and direct the audio data from the to the communication server 104. The audio data can be segmented into chunks, and each chunk may be associated with a respective notification and relayed accordingly, as described below.

Audio data recorded during the medical visit can be associated with a sequence identification number. For example, a first chunk or segment of audio data can be associated with a first sequence number, which may be a unique identifier of the audio data. In an embodiment, second audio data can be generated and the second audio data can be associated with a second sequence identification number. For example, the second audio data can correspond to the medical visit, e.g., a one minute segment of conversation immediately following a one minute segment of conversation recorded by the first chunk of audio data. The sequence identification numbers may be transmitted to the server along with the audio segments. More particularly, the sequence identification number associated with the first audio data can be transmitted to the communication server 104 with the audio data, and the second sequence identification number associated with the second audio data can be transmitted to the communication server 104 with the second sequence identification number.

In an embodiment, audio data that is stored in response to the notification indicating a transmission failure is not stored indefinitely. More particularly, the stored audio data can be deleted after a period of time. The period of time may be predetermined and may be selected to allow enough time for the connection to be reestablished, for example, when the physician moves to a more stable network connection. The period of time may, for example, be greater than 48 hours. Accordingly, the transmission of audio data may be attempted for a period of time after the medical visit concludes and, afterward, it may be assumed that the upload cannot be performed successfully and the recording may be deleted. This further clarifies that audio data can be cached, i.e., stored temporarily, and is not stored indefinitely or permanently.

As described above, the sequence identification numbers can uniquely identify the corresponding audio data. The sequence identification numbers can identify an order of the audio data chunks in a sequence of chunks representing the recorded conversation. For example, the sequence identification number of the first audio data can indicate that the audio data precedes the second audio data based on a value relative to the second sequence identification number. Accordingly, transmission of the audio data to the server can be based on the respective sequence identification number of the audio chunk. More particularly, the audio data and the second audio data are transmitted to the server in an order based on the sequence identification number and the second sequence identification number. Accordingly, when several chunks of audio data fail to successfully transmit to the server, the reattempt to send the chunks can be performed in the order defined by the sequence identification numbers of the chunks, in which chunks representing moments in time earlier than other chunks are reattempted before the other chunks. Transmission can therefore be attempted only for audio chunks that failed to transmit, and are reattempted in the order based on the respective sequence numbers. It will be appreciated that the server can receive the chunks of audio data in the order of successful transmission, however, irrespective of sequence identification number. For example, when a first chunk, a second chunk, and a third chunk are sent in sequence, the first chunk and third chunk may successfully transmit in a first attempt and the second chunk may successfully transmit in a subsequent second attempt. In such case, the server will receive the first chunk, then the third chunk, and then finally the second chunk.

Attempted transmission of audio data from the mobile device 101 to the communication server 104 can occur during the medical visit. For example, the audio data can be segmented, and segments may be communicated while the visit is being recorded prior to selection of the note element 206 (FIG. 2B). To generate an adequate note for the user, a certain amount of transcription may be required prior to the end of the medical visit. For example, if insufficient audio data has been successfully transmitted when the visit ends, then it may not be possible to provide a reliable summary of the visit immediately to the user. In an embodiment, a notification is generated when a predetermined percentage of audio data is not successfully received by the communication server 104 when the note element 206 is selected. For example, if at least 85% of the total audio data for the visit has not been successfully transmitted to the communication server 104 when the note element 206 is selected to end the recording, then the mobile device 101 may generate and display a notification to the user indicating that the generation of the note may be delayed or may be incomplete. Regardless of whether the notification is generated, the transmission attempts may continue for the period of time to eventually generate a reliable note for the user.

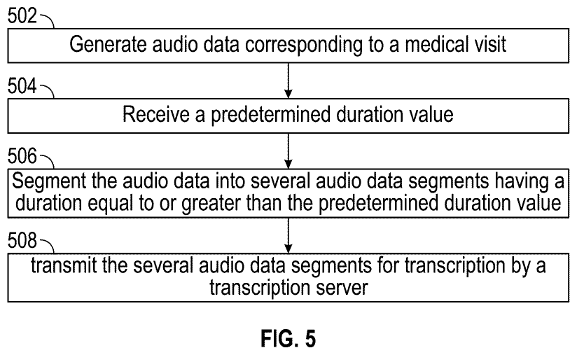

FIG. 5 is a flow diagram of operations performed by a mobile device during a method of transferring audio data in accordance with one or more embodiments of the disclosure. As described above, audio data recorded during the medical visit can be segmented, partitioned, or divided into chunks of audio data. Such audio segments, or audio chunks, can allow for audio file transmission and transcription to occur during and immediately after the medical visit, which can result in notes on the meeting being returned to the user soon, e.g., within 20 seconds, after selecting the note element 206 to end the medical visit. The immediate note return can create a perception of efficient performance of the system and method.

At operation 502, audio data is generated corresponding to the medical visit. The operation can be the same or similar to the process described above with respect to operation 302 of FIG. 3. Segmentation of the audio data can occur based on a duration setting. For example, at operation 504, the mobile device 101 can receive a duration value from the communication server 104. The duration value can be predetermined and can be a time value for an approximate length of each audio chunk for the mobile device 101 to transmit to the server at operation 304 of FIG. 3. For example, the duration value can be one minute, and may be communicated to the mobile device 101 prior to or after initiation of the recording.

The duration value may be selected based on several criteria. For example, one minute is a duration that approximates a period of time during conversation over which meaningful details of the conversation may emerge. Furthermore, in an embodiment, the duration value is based on a transcription speed at which the audio data can be transcribed by the transcription server 106. For example, the transcription server 106 may require 20 seconds to transcribe one minute of audio data and, thus, the duration value may be selected as one minute to ensure that partial transcripts are received within 20 seconds of the beginning of the recording. In any case, the duration value can be a value of more than 1 minute and/or less than 3 minutes, to reduce the likelihood that a corresponding partial transcript does not contain relevant information and to ensure that partial transcripts are quickly provided.

The duration value can be a minimum segment length of the audio chunks. More particularly, at operation 506, the audio data is recorded in portions that segment the audio data into several audio data segments having a duration equal to or greater than the duration value. When the duration value is reached, the mobile device 101 may determine, based on additional criteria, whether to conclude a portion of the recording to segment the audio data.

The additional criteria can include characteristics of the audio data that indicate whether the audio chunk contains an intelligible amount of data. In an embodiment, the additional criteria includes silence detection performed on the audio recording. More particularly, segmenting the audio data can include detecting a silence between the several audio data segments. Silence detection can determine whether a pause in speech is an end to a word or thought, or merely a transient pause in speech, such as between phonemes. For example, the silence detection parameters can include a predetermined noise level for a predetermined duration. The predetermined noise level may be possible to define because the mobile device 101 has an integrated microphone with known settings. More particularly, the predetermined noise level may be based on performance characteristics of the integrated microphone of the mobile device 101.

In an embodiment, the mobile device 101 detects the silence between the several audio data segments when the recorded audio data has a noise level less than 40 dB, e.g., 32 dB or less, for a duration of at least 100 milliseconds. Such a pause can indicate that a speaker has completed a word and is not speaking mid-word. As a results, the utterance “cancer” can be recorded in an audio chunk rather than being split into two audio chunks as “can” and “sir” when the word is spoken at the moment that the duration value of one minute is reached. It will be appreciated then that the combination of the duration value and the silence detection can be used to segment the audio data in a manner that creates audio chunks having intelligible amounts of data that lead to accurate transcripts of the conversation.

Silence can be searched for by the mobile device 101 during an entire time after the duration value. For example, after one minute elapses from an initiation of a current audio chunk, the mobile device 101 can search for, detect, and segment the audio chunk when the silence parameters are detected. In an embodiment, however, an upper limit may be placed on the length of each audio chunk. For example, an upper limit value following a beginning of the generated audio data for the current audio chunk can be set by the mobile device 101. The upper limit value may be, for example, 3 minutes. More particularly, the upper limit value can be set to a duration at which continuing noise levels above the silence parameter may be assumed to be ambient noises that are irrelevant to the content of the conversation. The audio chunk is therefore segmented at the upper limit value, e.g., 3 minutes, regardless of whether silence has been detected between the duration value and the upper limit value. Accordingly, the silence that causes segmentation of the audio chunk may therefore be searched for and occurs between the duration value and the upper limit value following the beginning of the generated audio data. If the silence does not occur within such time window, then the mobile device 101 may segment the audio data when the upper limit value is reached without regard to silence detection at that time.

At operation 508, the partitioned audio data segments are transmitted for transcription by the transcription server 106. The audio chunks can be sent by the mobile device 101 in real-time, e.g., during the medical visit, based on the sequence identification number described above. When the communication server 104 receives the audio chunks, the segments can be immediately, e.g., responsively, transmitted to the transcription server 106 to request transcripts. The transcription server 106 can return partial transcripts of the medical visit, e.g., transcripts of the audio chunks, to the communication server 104. The communication server 104 may then combine the partial transcripts based on the sequence identification numbers of the corresponding audio chunks to create an audio transcript of the medical visit.

The user can request a note, e.g., a transcript or a custom note related to the medical visit. For example, referring again to FIG. 2C, the user may select one of the note types to cause the mobile device 101 to request that a note be generated based on the audio transcript stored by the communication server 104.

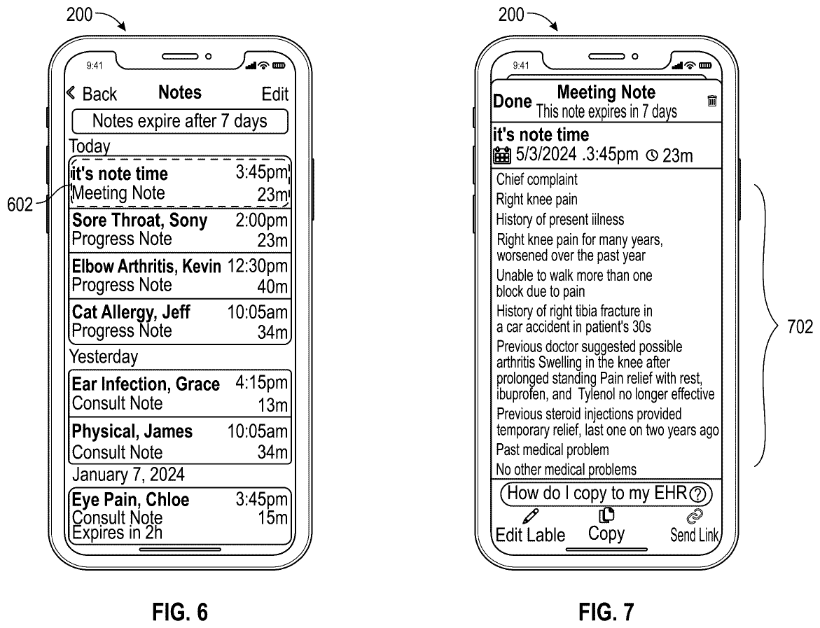

FIG. 6 is a pictorial view of a user interface for generating notes based on audio data recording a medical visit in accordance with an embodiment of the disclosure. When a note type is selected, the user can name and save the note into a list of notes stored on the mobile device 101. For example, the user may select a meeting note type and name the note “It's note time,” and the mobile device 101 can save the note as a note entry 602 in the list. The list can include other notes and note types, such as a progress note, a consult note, etc. Selection of the note entry 602 by the user, e.g., by tapping on the note entry 602, can transition the user interface 200 to a note screen that displays the note generated by the communication server 104 based on the audio data transcript.

FIG. 7 is a pictorial view of a user interface for displaying notes generated based on audio data recording a medical visit in accordance with an embodiment of the disclosure. The displayed note, which is displayed in the user interface 200, can include a summary of the medical visit based on the recorded audio. To generate the note, at the communication server 104, the transmitted audio data chunks are relayed to the transcription server 106. The transcription server 106 can generate transcripts of the conversation in each audio data chunk, and return the partial transcript to the communication server 104. The communication server 104 can store the partial transcripts for use. For example, when prompted by the mobile device 101, the communication server 104 may transmit the partial transcripts, or a full transcript assembled from the partial transcripts, to a Generative Pre-trained Transformer (GPT) model, e.g., a neural network-based language prediction models built on a transformer architecture, to analyze the prompt from the mobile device 101.

The transcripts can be supplied to the GPT model with a prompt. For example, the prompt can include a system prompt that is stored by the communication server 104 and provided with the transcript. The system prompt may include a reference to an imperfect transcription. More particularly, the system prompt may recognize that the transcript provided to the GPT model is not a perfectly accurate transcript of the medical visit. The system prompt can include, for example, “you are a medical assistant taking medically relevant notes for a medical visit based on an imperfect transcription of the visit.” The system prompt can be used by the GPT model to predict the best possible response using the supplied transcripts.

Note creation from transcribed audio can also be based on user prompts. For example, user prompts can facilitate generation of notes in a particular format. The resulting note, which is output by the GPT model and returned to the communication server 104 for use in generating the note that is then sent to and displayed on the mobile device 101, can summarize the medical visit. For example, as shown in FIG. 7, the note can include note content 702 summarizing a chief complaint of the patient, a history of present illness of the patient, and/or any past medical history, as discussed by the physician and the patient during the medical visit. The note content 702 can be formatted in a manner that can be readily saved to an electronic health record (EHR).

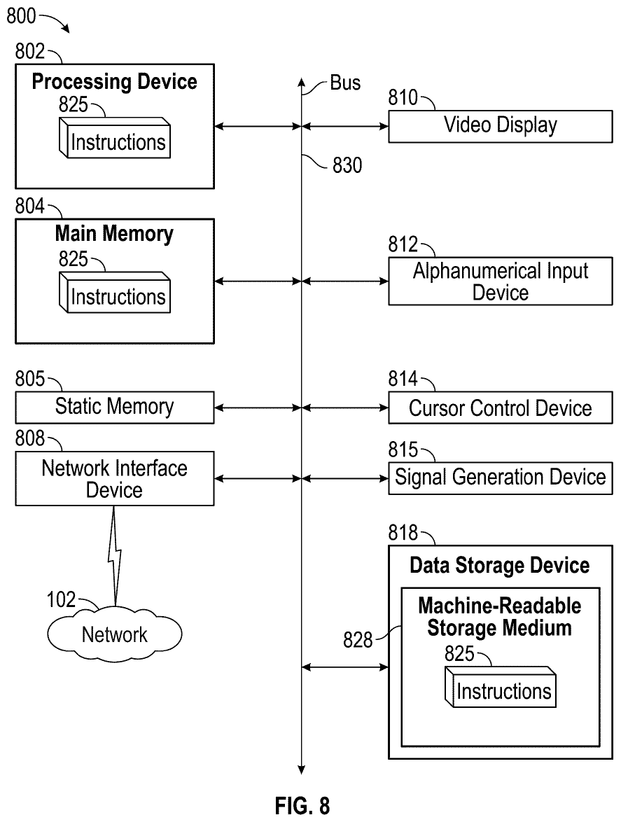

FIG. 8 is a block diagram of an example computing device that may perform one or more of the operations described herein, in accordance with some embodiments. More particularly, computing device 800 may be integrated in any of the servers and/or mobile devices described above to perform any of the described operations. Computing device 800 may be connected to other computing devices in a LAN, an intranet, an extranet, and/or the Internet. The computing device may operate in the capacity of a server machine in the client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 800 may include one or more processing devices 802 (e.g., a processing device, a general purpose processing device, a PLD, etc.), a main memory 804 (e.g., synchronous dynamic random access memory (DRAM), read-only memory (ROM)), a static memory 805 (e.g., flash memory and a data storage device), which may communicate with each other via a bus 830.

The one or more processing devices 802 may be provided by one or more general-purpose processing devices such as a microprocessor, central processing unit, or the like. In an illustrative example, processing device(s) 802 may comprise a complex instruction set computing (CISC) microprocessor, reduced instruction set computing (RISC) microprocessor, very long instruction word (VLIW) microprocessor, or a processing device implementing other instruction sets or processing devices implementing a combination of instruction sets. Processing device(s) 802 may also comprise one or more special-purpose processing devices such as an application specific integrated circuit (ASIC), a field programmable gate array (FPGA), a digital signal processor (DSP), network processor, or the like. The processing device(s) 802 may be configured to execute the operations described herein, in accordance with one or more aspects of the present disclosure, for performing the operations and steps discussed herein.

Computing device 800 may further include a network interface device 808 which may communicate with the network 102. The computing device 800 also may include a video display unit 810 (e.g., a liquid crystal display (LCD) or a cathode ray tube (CRT)), an alphanumeric input device 812 (e.g., a keyboard), a cursor control device 814 (e.g., a mouse) and an acoustic signal generation device 815 (e.g., a speaker). In one embodiment, video display unit 810, alphanumeric input device 812, and cursor control device 814 may be combined into a single component or device (e.g., an LCD touch screen).

Data storage device 818 may include a non-transitory computer-readable storage medium 828 on which may be stored one or more sets of instructions 825 that may include instructions for carrying out the operations described herein, in accordance with one or more aspects of the present disclosure. Instructions 825 may also reside, completely or at least partially, within main memory 804 and/or within processing device(s) 802 during execution thereof by computing device 800, main memory 804 and processing device(s) 802 also constituting computer-readable media. The instructions 825 may further be transmitted or received over a network 820 via network interface device 808.

While computer-readable storage medium 828 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding, or carrying a set of instructions for execution by the machine or system and that cause the machine or system to perform the methods described herein. The term “computer-readable storage medium” shall accordingly be taken to include, but not be limited to, solid-state memories, optical media, and magnetic media.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

What is claimed is:

1. A method, comprising:
generating audio data corresponding to a medical visit;
receiving a duration value; and
segmenting, by a processing device, the audio data into a plurality of audio data segments having a duration equal to or greater than the duration value.

2. The method of claim 1 further comprising transmitting the plurality of audio data segments for transcription by a transcription server, wherein the duration value is based on a transcription speed at which the audio data can be transcribed by the transcription server.

3. The method of claim 1, wherein the duration value is received prior to generating the audio data.

4. The method of claim 1, wherein segmenting the audio data includes detecting a silence between the plurality of audio data segments.

5. The method of claim 4, wherein the silence is detected when the audio data has a noise level less than 32 dB for at least 100 milliseconds.

6. The method of claim 4, wherein the silence occurs between the duration value and an upper limit value following a beginning of the generated audio data.

7. The method of claim 6, wherein the duration value is 1 minute.

8. The method of claim 6, wherein the upper limit value is 3 minutes.

9. A system comprising:
a memory to store instructions; and
a processing device to execute the instructions to cause the system to:
generate audio data corresponding to a medical visit,
receive a duration value, and
segment the audio data into a plurality of audio data segments having a duration equal to or greater than the duration value.

10. The system of claim 9, wherein the processing device is further to execute the instructions to cause the system to:
transmit the plurality of audio data segments for transcription by a transcription server, wherein the duration value is based on a transcription speed at which the audio data can be transcribed by the transcription server.

11. The system of claim 9, wherein the duration value is received prior to generating the audio data.

12. The system of claim 9, wherein segmenting the audio data includes detecting a silence between the plurality of audio data segments.

13. The system of claim 12, wherein the silence is detected when the audio data has a noise level less than 40 dB for at least 100 milliseconds.

14. The system of claim 12, wherein the silence occurs between the duration value and an upper limit value following a beginning of the generated audio data.

15. A non-transitory computer-readable medium storing one or more instructions that, when executed by a processing device of a system, cause the system to:
generate audio data corresponding to a medical visit;
receive a duration value; and
segment, by the processing device, the audio data into a plurality of audio data segments having a duration equal to or greater than the duration value.

16. The non-transitory computer-readable medium of claim 15, wherein the one or more instructions, when executed by the processing device, cause the system to transmit the plurality of audio data segments for transcription by a transcription server, wherein the duration value is based on a transcription speed at which the audio data can be transcribed by the transcription server.

17. The non-transitory computer-readable medium of claim 15, wherein the duration value is received prior to generating the audio data.

18. The non-transitory computer-readable medium of claim 15, wherein segmenting the audio data includes detecting a silence between the plurality of audio data segments.

19. The non-transitory computer-readable medium of claim 18, wherein the silence is detected when the audio data has a noise level less than 40 dB for at least 100 milliseconds.

20. The non-transitory computer-readable medium of claim 18, wherein the silence occurs between the duration value and an upper limit value following a beginning of the generated audio data.
